(19) United States
(12) Patent Application Publication
(10) Pub. No.: US 2015/0140121 A1
(43) Pub. Date: May 21, 2015

(54) Compositions and Methods for Tight Junction Modulation

(71) Applicants: Lankenau Institute for Medical Research, Wynnewood, PA (US); Monk Street Partners, LLC., Collegeville, PA (US)

(72) Inventors: James M. Mullin, Havertown, PA (US); Jonathan M. Raines, Gladwyne, PA (US)

(21) Appl. No.: 14/406,451

(22) PCT Filed: Jun. 10, 2013
(86) PCT No.: PCT/US13/44966

§ 371 Date: Dec. 8, 2014

Related U.S. Application Data
(60) Provisional application No. 61/657,324, filed on Jun. 8, 2012.

(51) Int. Cl. A61K 33/30; A61K 31/352; G01N 33/50; A61K 31/404

(52) U.S. Cl. CPC A61K 33/30; A61K 31/404; A61K 31/352; G01N 33/5032; G01N 2500/10
USPC 424/642; 424/641; 424/643; 435/29

(57) ABSTRACT

Compositions and methods for modifying the composition of tight junction complexes and/or improving epithelial barrier function are disclosed.

This application claims priority under 35 U.S.C. §119(e) to U.S. Provisional Patent Application No. 61/657,324, filed on Jun. 8, 2012. The foregoing application is incorporated by reference herein.

### FIELD OF THE INVENTION

The present invention relates to the field of epithelial barriers and tight junction control. Specifically, compositions and methods for modulating tight junctions and the effectiveness of epithelial barriers are disclosed, toward the purpose of controlling progression of epithelial diseases (e.g., disease due to transepithelial leak and/or pathogen utilization of tight junction complexes).

### BACKGROUND OF THE INVENTION

Several publications and patent documents are cited throughout the specification in order to describe the state of the art to which this invention pertains. Each of these citations is incorporated herein by reference as though set forth in full.

Any epithelial tissue layer, be it the lining of the lungs, gall bladder, mammary gland, stomach, colon, urinary bladder, or the like has two principal components to prevent unrestricted and harmful leak across the tissue layer: the epithelial cells themselves and the gasket-like junctional sealing strands (the “tight junction” [TJ]) that circumferentially bands each epithelial cell at its apical pole (Schneeberger et al. (2004) Am. J. Physiol. Cell Physiol., 286:C1213-28; Furuse, M. (2010) Cold Spring Harb. Perspect. Biol., 2:a002907). This barrier function is essential physiologically for preventing back-leak of substances that the epithelial cells have pumped across the tissue layer. Secondly, it is critical immunologically for preventing noxious substances in epithelial tissue luminal compartments from crossing the lining and gaining access to the bloodstream, with chronic inflammation, sepsis, or cancer progression the possible results.

Epithelial barriers are not fixed, static structures. They can become more leaky or less leaky in response to a wide array of different stimuli. Disease processes and agents almost uniformly act to make barrier linings leakier, and the site of such leak is generally at the TJ (Mullin et al. (2005) Drug Discov. Today, 10:395-408). Bacterial toxins, viruses, cancer, diabetes and mere inflammation can all generate leakiness in the epithelial tissues that they affect.

Because TJ permeability is a dynamic phenomenon, increasing to one stimulus and/or decreasing to another, the basal state of TJs in any given epithelial tissue can generally be modulated to become tighter or modulated to become leakier. In general, increased leakage is deleterious, while decreased leakage works either physiologically or immunologically to the organism's benefit. Improved compositions and methods for the modulation (e.g., tightening) of tight junctions are desired.

### SUMMARY OF THE INVENTION

In accordance with the present invention, methods of altering the structure/composition of tight junctions and/or improving/increasing tight junction barrier function (e.g., increasing transepithelial electrical resistance; decreasing leakage of molecules across the barrier; and/or inhibiting microbial passage across the barrier) in an epithelial layer/sheet are provided. More particularly, the instant invention provides methods of altering tight junctions structurally and/or improving epithelial barrier function comprising administering at least two agents which decrease leakage/passage across tight junctions to the epithelial tissue. In a particular embodiment, the more than one agent produces an additive effect, particularly a synergistic effect, to alter/improve tight junction barrier function. The instant invention also encompasses methods of inhibiting, treating, and/or preventing a disease or disorder associated with epithelial barrier leakage. In a particular embodiment, the method comprises administering at least two agents which decrease leakage/passage across tight junctions to the epithelial tissue of a subject.

In accordance with the instant invention, methods of screening for at least additive, particularly synergistic, combinations of agents for improving epithelial barrier function (e.g., increasing transepithelial electrical resistance; decreasing leakage of molecules across the barrier; and/or inhibiting microbial passage across the barrier) are provided. In a particular embodiment, the method comprises a) contacting an epithelial layer with at least two different agents for decreasing leakage across tight junctions; and b) measuring the leakage of the epithelial layer, wherein a decrease in the leakage measured in step b) compared to the leakage observed with the agents individually indicates a synergy with the tested agents. In a particular embodiment, the screening methods identify agents which work synergistically against a particular disease or disorder.

### BRIEF DESCRIPTIONS OF THE DRAWING

FIG. 1 provides a schematic of the gastrointestinal epithelial barrier.

FIG. 2 provides a diagrammatic representation of the tight junction complex in the epithelial barrier.

FIG. 3A shows the relative expression of Claudin 2 in LLC-PK1 cell layers that were untreated (control) or treated with 400 μM quercetin. FIG. 3B shows the relative expression of Claudin 5 in LLC-PK1 cell layers that were untreated (control) or treated with 400 μM quercetin.

FIG. 4A provides a graph of transepithelial resistance of untreated (control) LLC-PK1 cell layers or LLC-PK1 cell layers treated with 100 μM, 200 μM, or 400 μM quercetin. n=9. Student's t test relative to control is provided. FIG. 4B provides a graph of mannitol leak across untreated (control) LLC-PK1 cell layers or LLC-PK1 cell layers treated with 100 μM, 200 μM, or 400 μM quercetin. n=6. P<0.05 for the 400 μM quercetin.

FIG. 5A shows the relative expression of Claudin 4 in LLC-PK1 cell layers that were untreated (control) or treated with 1 mM indole. FIG. 5B shows the relative expression of Claudin 5 in LLC-PK1 cell layers that were untreated (control) or treated with 1 mM indole.

FIG. 6 provides a graph of transepithelial resistance of untreated (control) LLC-PK1 cell layers or LLC-PK1 cell layers treated with 0.1 mM, 1 mM, or 2 mM indole.

FIG. 7A provides a graph of transepithelial resistance of untreated (control) LLC-PK1 cell layers or LLC-PK1 cell layers treated with 50 μM, 100 μM, or 200 μM zinc. FIG. 7B provides a graph of ¹⁴C-D-mannitol leak across untreated (control) LLC-PK1 cell layers or LLC-PK1 cell layers treated with 50 μM, 100 μM, or 200 μM zinc.

FIG. 8 provides a graph of transepithelial resistance of untreated (control) LLC-PK1 cell layers or LLC-PK1 cell layers treated with 100 μM zinc, 400 μM quercetin, or 100 μM zinc with 400 μM quercetin.

### DETAILED DESCRIPTION OF THE INVENTION

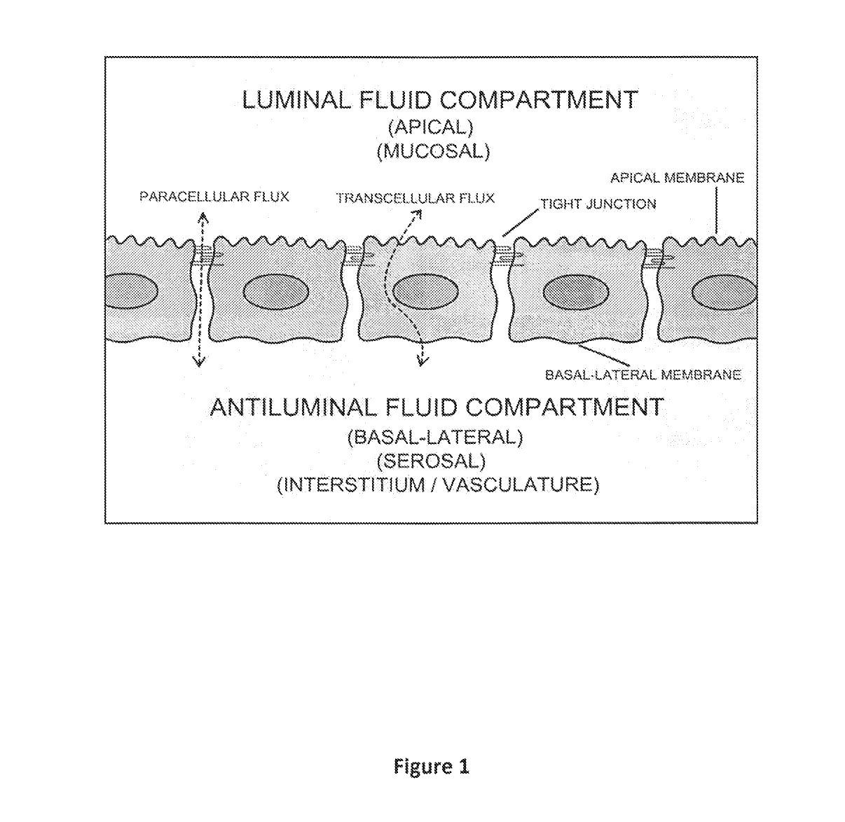

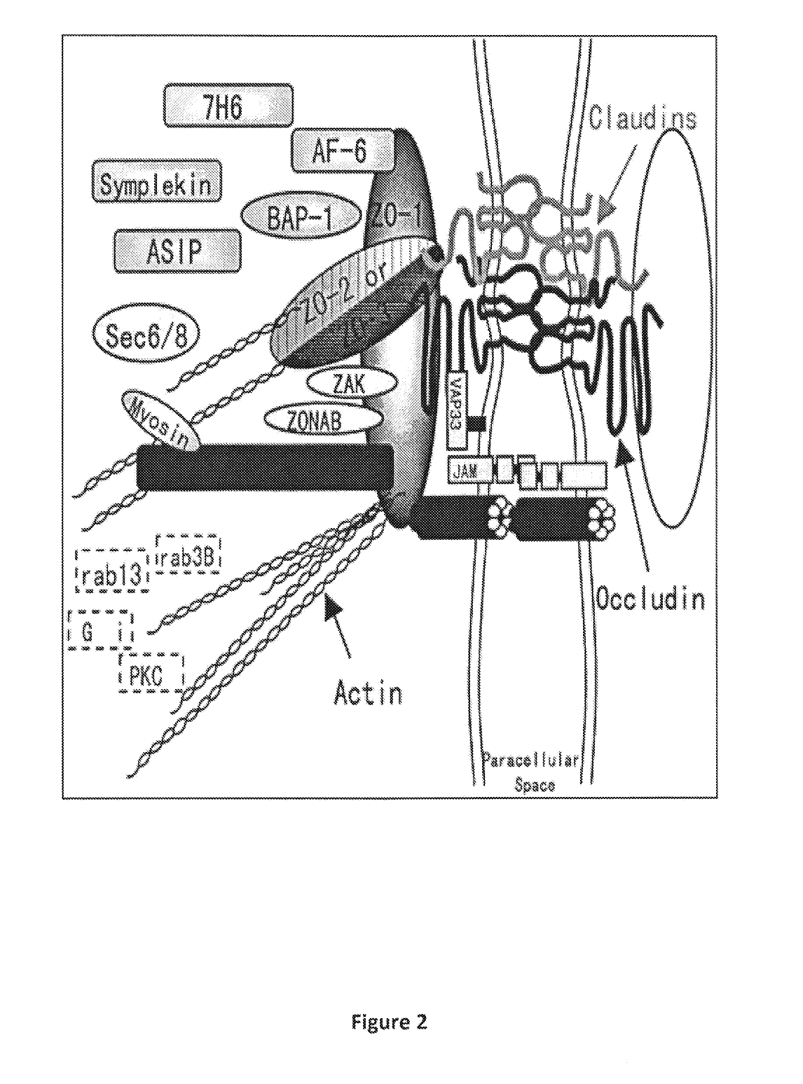

The epithelial barrier comprises epithelial cells that are circumferentially banded by tight junctions at their apical pole (see, e.g., FIG. 1). The epithelial or endothelial TJ is a complex structure (see FIG. 2). In terms of composition it can be divided into two sets of proteins. First there are the integral membrane proteins that make up the physical barrier. These proteins are embedded in the plasma membrane of a cell and physically link up with the same proteins on an adjoining cell to generate the TJ barrier. These proteins include occludin, tricellulin and various representatives of the 26-member claudin family. Second, there are the TJ-associated proteins, all located in the cell, just inside the plasma membrane. These proteins provide a physical link between the barrier proteins and the cell's actin cytoskeleton. This group includes ZO-1, ZO-2, ZO-3, 7H6, AF-6, and the like.

Each of these proteins represents a potential different target by which a signaling mechanism can cause a change in TJ permeability. For example, phosphorylation of ZO-1, up-regulation of claudin-5, down-regulation of claudin-2, can each cause unique changes to the permeability of the TJ. Depending upon the specific epithelial cell layer, leakiness may be increased or decreased by the agent used and the protein targeted. For example, upregulation of claudin-5 might decrease leakiness, whereas phosphorylation of occludin might increase leakiness. The compositional complexity of these junctions leads to a wide array of possibilities for affecting permeability.

In addition to the above, the degree of leakiness of the TJ must be considered along with the substrate specificity of the leakiness. The compositional complexity of TJs means that changes in their permeability can be highly specific. For example, certain TJs may become leaky only to salts—very small but electrically charged molecules—in response to a particular stimulus. A different type of stimulus may lead to TJs becoming leaky not to salts, but to small, uncharged molecules such as simple sugars. A third type of stimulus might lead to effective TJ leakiness only to large molecules (e.g. small proteins or small fragments of bacterial toxins). This last scenario—wherein leakiness to large molecules and not to small molecules occurs—may seem counterintuitive. However, the sites of large molecule leak are very sparse in a cell layer whereas small molecule leaks may occur essentially the full length of the TJ bands. Finally, a stimulus may also induce leaks to all types of molecules as well as water. Agents which induce leakiness in TJs include, without limitation, heavy metals (e.g., cadmium), viruses, bacteria, bacterial toxins, lipidic signaling molecules (e.g., phorbol esters and diacylglycerols), chelating agents, inflammatory mediators (e.g., tumor necrosis factor), and the like.

As with agents that induce leakiness in TJs, agents that induce TJ ‘tightening’ or structurally change TJs can likewise induce TJ permeability changes for one or more specific class of molecules. Agents with induce TJ tightening and/or structurally alter TJs include, without limitation: zinc, certain amino acids (e.g., glutamine and arginine), rapamycin, berberine, flavonoids (e.g., quercetin, genistein, epigallocatechin, see also Noda et al. (J. Agric. Food Chem.; PMID: 22506771), isoflavanoids, lycopenes, stilbenoids (e.g., resveratrol), cruciform-derived compounds (e.g., organo-sulfur compounds such as sulforaphane), anthocyanins (e.g., cyanidin), short chain and omega-3 fatty acids and lipids (e.g., linolenic acid, lipoic acid, and butyrate), indole, selenium, niacin, vitamin D, nicotine, and probiotics. Notably, the agents are all naturally occurring and, for the most part, are nutritional in nature.

Each of the above TJ tightening agents may affect TJs at different target sites and achieve a different kind of tightening. This specificity of action, both structurally and functionally, indicates that combinations of the above agents can synergize and create a TJ barrier tightening that is superior to that which can be achieved by either agent alone. For example, for a particular disease and for a particular affected tissue, treating the tissue with zinc may achieve TJ ‘tightening’ that reduces leakage of salts and water (edema), but may not address the leakage to bacterial toxins that is inducing inflammation. However, the co-treatment of zinc with another agent such as indole or rapamycin can reduce the leakage of the bacterial toxin, thereby achieving a synergistic effect with zinc and more fully alleviating the disease.

The actions of certain individual agents capable of barrier enhancement on TJ permeability are described below. As stated hereinabove, each agent has different TJ targets and unique effects.

### 1. Zinc

Zinc has long been known to be efficacious in the treatment of certain types of diarrhea. It has been determined that zinc can render certain TJ barriers less leaky. Zinc effects can vary depending upon the specific type of epithelial layer. Exposure of a renal proximal tubular-like (LLC-PK1) epithelium to zinc at 50 and 100 micromolar concentrations resulted in two aspects of TJ ‘tightening’: 1) increased transepithelial electrical resistance (Rt); and 2) decreased transepithelial diffusion of the uncharged sugar alcohol, D-mannitol. However when the TJ proteins occludin, tricellulin and claudins 1 through 5 were examined by Western immunoblot, none had exhibited increased or decreased expression, indicating either some other TJ protein was effecting the permeability change of the tight junction and/or a change in the above TJ proteins other than their net amount or a shift in their cellular localization (e.g., a phosphorylation or dephosphorylation).

When the effects of 50 or 100 micromolar zinc were examined on the TJ permeability of a human colonic (CACO-2) epithelial cell layer, the results were quite different. Here the Rt increased as with the renal epithelium. However, there was an increase—as opposed to a decrease—in a D-mannitol diffusion. In other words, zinc decreased leak to salts but increased leak to small nonelectrolytes. On the structural level, significant decreases in claudins 2 and 7 and no changes in occludin, tricellulin or claudins 1, 3, 4 and 5 amounts were observed with the human colonic (CACO-2) epithelial cell layer and, as explained hereinbelow, no changes in amounts of these proteins were observed with a LLC-PK1 renal epithelial model. Even though the overall cellular content of the tight junction protein(s) may not be affected, zinc may cause a ‘shift’ in certain tight junction protein (e.g., certain claudins) out of the tight junctional complex and into the cell's cytoplasm. This “shift” has been observed with claudins 2 and 4 in the LLC-PK1 renal epithelial model. Zinc can also regulate the localization of both occludin and ZO-1 in colonic (CACO-2) epithelia (Finamore et al. (2008) J. Nutr., 138:1664-70).

In small intestine, zinc supplementation increased levels of occludin and ZO-1 while decreasing leakage of the small non-electrolyte lactulose (Zhang et al. (2009) Br. J. Nutr., 102:687-93).

### 2. Amino Acids (e.g., Glutamine/Arginine)

Glutamine, a non-essential amino acid, has been observed to improve barrier function in both isolated colonic (CACO-2) epithelia as well as small intestinal tissue (Ewaschuk et al. (2011) Br. J. Nutr., 106:870-7; Li et al. (2009) J. Nutr., 139:710-4). In the CACO-2 epithelial cell layers, glutamine increased Rt and decreased mannitol diffusion while also increasing claudin-1 expression (Li et al. (2009) J. Nutr., 139:710-4). Glutamine has also been shown to reverse toxixicity-related increases in TJ leakiness, an effect related to redistribution of the TJ proteins, occludin and ZO-1 (Seth et al. (2004) Am. J. Physiol. Gastrointest. Liver Physiol., 287:G510-7). The amino acid, arginine, exerts very similar barrier-enhancing effects (Rhoads et al. (2009) Amino Acids 37:111-22).

### 3. Quercetin

The flavonoid quercetin at a concentration of 0.2 mM, increased Rt across colonic (CACO-2) epithelial cell layers due to decreased TJ permeability to Na⁺ and Cl⁻ ions, but had no significant effect on TJ permeability to the nonelectrolyte D-mannitol (Amasheh et al. (2008) J. Nutr., 138:1067-73). Accompanying this mode of barrier enhancement was a dramatic increase in the TJ content of claudin-4. The amounts of occludin and claudins 1, 3 and 7 were unaffected. In contrast, as explained hereinbelow, the expression of certain claudins is greatly increased or decreased in a LLC-PK1 renal epithelial model. In an independent study, the increase in claudin-4 was confirmed, but a quercetin-induced cellular redistribution of claudin-1, occludin and ZO-2 also occurred (Suzuki et al. (2009) J. Nutr., 139:965-74). The electrically charged dye, Lucifer yellow, also had its permeability decreased by quercetin treatment.

### 4. Indole

The bacterial tryptophan metabolite indole at a concentration of 1 mM, increased Rt and decreased the abundance of claudin-2 across human colonic (HCT-8) epithelial cell layers (Bansal et al. (2010) Proc. Natl. Acad. Sci., 107:228-33).

### 5. Selenium

Selenium at a supplemental concentration (e.g., 100 ng/ml), increased Rt and also decreased paracellular invasion of cancer cells across a mammary epithelial barrier (Martin et al. (2007) J. Cell Biochem., 101:155-66). These functional changes were accompanied by cellular redistribution of the TJ proteins, ZO-1 and occludin, from cytosolic to junctional regions of the cell.

### 6. Linolenic Acid

The omega-3 fatty acid linolenic acid had similar enhancing effects on permeability and TJ proteins to those of selenium (Martin et al. (2007) J. Cell Biochem., 101:155-66). Other omega-3 fatty acids have protective effects on epithelial TJs (Li et al. (2008) Mol. Immunol., 45:1356-65).

### 7. Butyrate

The short chain fatty acid butyrate at 2 mM concentration both increased Rt and decreased paracellular leak of the large nonelectrolyte, inulin, in human colonic (CACO-2) cell layers. Although amounts of the TJ proteins, ZO-1, occludin, claudin-1 and claudin-4 were unaffected by butyrate, there was induced cellular redistribution of ZO-1 and occludin (Peng et al. (2009) J. Nutr., 139:1619-25). Butyrate and other short chain fatty acids exert similar tightening effects on human endothelial cell layers (Miyoshi et al. (2008) Nutrition 24:1189-98).

### 8. Rapamycin

Concentrations of rapamycin as low as 1 micromolar stimulated expression of claudins 1, 4 and 7 in renal epithelial (LLC-PK1) cell layers and also increased Rt (Martin-Martin et al. (2010) Am. J. Physiol. Renal Physiol., 298:F672-82).

### 9. Berberine

Berberine (50 micromolar) prevents the TJ leakiness and occludin redistribution in colonc epithelial (CACO-2 and HT-29/B6) cell layers that ensues from proinflammatory cytokine exposure (Li et al. (2010) Eur. J. Pharm. Sci., 40:1-8; Amasheh et al. (2010) J. Cell Sci., 123:4145-55). Strong effects of berberine on the cellular amounts of claudins 1 and 2 were observed, but there was no effect of berberine on the amounts of occludin or claudins 3, 4, 5 or 7.

In accordance with the instant invention, methods of improving epithelial (e.g., endothelial) barriers are provided. In a particular embodiment, the method decreases the leakage (e.g., the traversal of molecules and/or microbes) across the epithelial barrier. The instant invention encompasses methods of inhibiting (e.g., reducing, suppressing), preventing, and/or decreasing tight junction leakage (e.g., increasing TJ barrier function and/or increasing transepithelial electrical resistance) in an epithelial layer/sheet. The methods may lead to the alteration of the composition of the tight junction (e.g., lead to the down-regulation of a protein (e.g., receptor; e.g., claudin (e.g., claudin 5)) that facilitates entry of a pathogen, bacteria or virus into the epithelial cell and/or the blood stream). In a particular embodiment, the method comprises administering at least two, at least three, at least four, at least five, at least six, at least seven, or more different agents that restrict passage across tight junctions (e.g., “tightens” tight junctions; see agents above). The agents administered may be, for example, selected from the group consisting of zinc, amino acids (e.g., glutamine and/or arginine), flavonoids (e.g., quercetin), anthocyanins, isoflavonoids, stillbenoids, organo-sulfur compounds derived from cruciforms, lycopenes, indole, selenium, omega-3 fatty acids (e.g., linolenic acid), short chain fatty acids (e.g., butyrate), rapamycin (sirolimus), berberine, and niacin.

In a particular embodiment of the instant invention, agents with different specificity of action on the epithelial barrier to be treated are co-administered. In a particular embodiment, at least one agent which inhibits leakage of salts is co-administered with at least one agent which inhibits the leakage of non-electrolyes (either small (e.g., mannitol or lactulose) or large (e.g., insulin)), and/or charged compounds/molecules. For example, on colonic epithelial cells, zinc decreases salt transfer but increases the leak to small nonelectrolytes such as mannitol. In contrast, glutamine administration to colonic epithelial cells decreased mannitol diffusion across the cell barrier. Therefore, in accordance with the instant invention, zinc and glutamine can be co-administered to colon epithelial cells in order to synergistically improve colonic epithelial barriers and reduce leakage across the barrier. Other examples of synergistic combinations, particularly on colonic epithelial, include, without limitation, zinc with amino acids (e.g., arginine), flavonoids (e.g., quercetin), and/or short chain fatty acids (e.g., butyrate). While combinations with zinc have been specifically exemplified, combinations of other agents in the absence of zinc (e.g., glutamine with quercetin) are also encompassed by the instant invention.

In a particular embodiment of the instant invention, at least one of the tight junction “tightening” agents delivered to the epithelial cells is zinc. The zinc may be administered as a complex with another compound. In a particular embodiment, at least one pharmaceutically acceptable salt of zinc is administered. Zinc salts include, without limitation, a zinc acetate, zinc butyrate, zinc gluconate, zinc glycerate, zinc glycolate, zinc formate, zinc lactate, zinc picolinate, zinc propionate, zinc salicylate, zinc tartrate, zinc undecylenate, zinc oxide, zinc stearate, zinc citrate, zinc phosphate, zinc carbonate, zinc borate, zinc ascorbate, zinc benzoate, zinc bromide, zinc caprylate, zinc carnosine, zinc chloride, zinc fluoride, zinc fumarate, zinc gallate, zinc glutarate, zinc glycerophosphate, zinc hydroxide, zinc iodide, zinc malate, zinc maleate, zinc myristate, zinc nitrate, zinc phenol sulfonate, zinc picrate, zinc propionate, zinc selenate, zinc succinate, zinc sulfate, zinc titanate, and zinc valerate. In a particular embodiment, the zinc is administered as complexed with gluconate (zinc gluconate).

The compounds of the instant invention (e.g., agents which improve barrier function) may be contained within a single composition comprising at least one pharmaceutically acceptable carrier. Alternatively, the compounds may be contained in separate compositions comprising at least one pharmaceutically acceptable carrier. The instant invention also encompasses kits comprising at least one composition as described herein, particularly wherein there at least two compositions. The compounds of the instant invention may be administered simultaneously and/or sequentially. For example, a second agent may be administered before, after, and/or simultaneously with zinc.

“Pharmaceutically acceptable” refers to entities and compositions that are physiologically tolerable and do not typically produce an allergic or similar untoward reaction when administered to an animal, particularly a human. Pharmaceutically acceptable carriers are preferably approved by a regulatory agency of the Federal or a state government or listed in the U.S. Pharmacopeia or other generally recognized pharmacopeia for use in/on animals, and more particularly in/on humans.

A “carrier” refers to, for example, a diluent, adjuvant, excipient, auxiliary agent, preservative, solubilizer, emulsifier, adjuvant, stabilizing agent or vehicle with which an active agent of the present invention is administered. Common carriers include, without limitation, sterile liquids, water (e.g., deionized water), alcohol (e.g., ethanol, isopropanol), oils (including those of petroleum, animal, vegetable or synthetic origin, such as peanut oil, soybean oil, mineral oil, sesame oil and the like), Common carriers include, without limitation, water, aqueous solutions, aqueous saline solutions, aqueous dextrose solutions, aqueous glycerol solutions, oil, buffered saline, ethanol, polyol (for example, glycerol, propylene glycol, liquid polyethylene glycol and the like), dimethyl sulfoxide (DMSO), detergents, suspending agents, glucose, lactose, gum acacia, gelatin, mannitol, starch paste, magnesium trisilicate, talc, corn starch, keratin, colloidal silica, potato starch, urea, medium chain length triglycerides, dextrans, other organic compounds or copolymers and other carriers suitable for use in manufacturing preparations, in solid, semisolid, or liquid form, and suitable mixtures thereof. Suitable pharmaceutical carriers and other agents of the compositions of the instant invention are described in “Remington's Pharmaceutical Sciences” by E. W. Martin (Mack Pub. Co., Easton, Pa.) and “Remington: The Science And Practice Of Pharmacy” by Alfonso R. Gennaro (Lippincott Williams & Wilkins). The compositions can include diluents of various buffer content (e.g., Tris HCl, acetate, phosphate), pH and ionic strength; and additives such as detergents and solubilizing agents (e.g., Tween 80, Polysorbate 80), anti oxidants (e.g., ascorbic acid, sodium metabisulfite), preservatives (e.g., Thimersol, benzyl alcohol) and bulking substances (e.g., lactose, mannitol). The pharmaceutical composition of the present invention can be prepared, for example, in liquid form, or can be in dried powder form (e.g., lyophilized).

The composition(s) may be a time release formulation. For example, the compositions can also be incorporated into particulate preparations of polymeric compounds such as polyesters, polyamino acids, hydrogels, polylactide/glycolide copolymers, ethylenevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (see, e.g., Remington's Pharmaceutical Sciences (Mack Publishing Co., Easton, Pa.; Medical Applications of Controlled Release, Langer and Wise (eds.), CRC Press: Boca Raton, Fla.; Controlled Drug Bioavailability, Drug Product Design and Performance, Smolen and Ball (eds.), Wiley: New York; Ranger and Peppas (1983) J. Macromol. Sci. Rev. Macromol. Chem., 23:61; Levy et al., Science (1985) 228:190; During et al. (1989) Ann. Neurol., 25:351; Howard et al. (1989) J. Neurosurg., 71:105).

The compositions of the present invention can be administered by any suitable route. The composition may be administered systemically or directly (locally) to a desired site. In a particular embodiment, the compositions are prepared for topical administration. The composition may be administered by any suitable means including, without limitation, topical, epidermal, oral, ocular (e.g., corneal), intrarectal, inhalation, pulmonarily, intranasal, and intravaginal administration. The agents may be delivered to, for example, the skin or oral, colorectal, bladder, uterine, nasal, vaginal, penile, nasopharyngeal, buccal, or intestinal epithelial or mucosa.

The composition for topical administration may be formulated, for example, as a suppository, enema, cream, lotion, foam, ointment, liquid, powder, salve, gel (e.g., intravaginal gel), milky lotion, drops, stick, spray (e.g., pump spray, feminine or masculine deodorant sprays), aerosol, paste, mousse, douche, or dermal patch. Types of pharmaceutically acceptable topical carriers include, without limitation, emulsions (e.g., microemulsions and nanoemulsions), gels (e.g., an aqueous, alcohol, alcohol/water, or oil (e.g., mineral oil) gel using at least one suitable gelling agent (e.g., natural gums, acrylic acid and acrylate polymers and copolymers, cellulose derivatives (e.g., hydroxymethyl cellulose and hydroxypropyl cellulose), and hydrogenated butylene/ethylene/styrene and hydrogenated ethylene/propylene/styrene copolymers), solids (e.g., a wax-based stick, soap bar composition, or powder (e.g., bases such as talc, lactose, starch, and the like), and liposomes (e.g., unilamellar, multilamellar, and paucilamellar liposomes, optionally containing phospholipids). The pharmaceutically acceptable carriers also include stabilizers, penetration enhancers (see, e.g., Remington's), chelating agents (e.g., EDTA, EDTA derivatives (e.g., disodium EDTA and dipotassium EDTA), iniferine, lactoferrin, and citric acid), and excipients. Protocols and procedures which facilitate certain formulation of the topical compositions can be found, for example, in Cosmetic Bench Reference 2005, Published by Cosmetics & Toiletries, Allured Publishing Corporation, Illinois, USA, 2005 and in International cosmetic ingredient dictionary and handbook. 10th ed. Edited by Tatra E. Gottschalck and Gerald E. McEwen. Washington, Cosmetic, Toiletry and Fragrance Association, 2004.

In a particular embodiment, the composition is administered orally. The composition for oral administration may be formulated as a pill, powder, capsule, caplet, tablet (e.g., coated and uncoated, chewable), gelatin capsule (e.g., soft or hard), time-release capsule, lozenge, troche, liquid solution (e.g., gargle), buccal strips or tablets, emulsion, suspension, syrup, elixir, powders/granules (e.g., reconstitutable or dispersible), aerosolized spray, ointment, or gum. Compositions for oral administration may comprise thickeners, flavorings, diluents, emulsifiers, dispersing aids or binders.

In a particular embodiment, the agents of the instant invention are delivered via a device (e.g., stent) or applicator to the epithelial tissue. For example, the topical compositions may be applied by an applicator such as a wipe, swab, or roller. In a particular embodiment, the zinc of the instant invention is applied to or incorporated into contraceptive devices such as a condom, diaphragm, cervical cap, intrauterine device (IUD), or vaginal sponge (e.g., contraceptive sponge). The zinc may also be administered via an implantable device such as a luminal stent, tube, or ring. The implantable medical device may be coated with a composition comprising zinc or may elute the composition. In a particular embodiment, the stent is dissolvable or degradable (e.g., a stent that exhibits substantial mass or density reduction or chemical transformation after it is introduced into a subject). In another embodiment, the stent is removable. The stent may be a sustained release device. Examples of esophageal stents include, without limitation, the Boston Scientific Ultraflex™ device, the Medtronic EsophaCoil® device, and the Cook Medical Evolution® device.

The agents described herein will generally be administered to a patient as a pharmaceutical preparation. The term “patient” as used herein refers to human or animal subjects. The compositions of the instant invention may be employed therapeutically, under the guidance of a physician, veterinarian, or other healthcare professional.

The compositions comprising the agent(s) of the instant invention may be conveniently formulated for administration with any pharmaceutically acceptable carrier(s). The concentration of the agent in the chosen medium may be varied and the medium may be chosen based on the desired route of administration of the pharmaceutical preparation. Except insofar as any conventional media or agent is incompatible with the agent(s) to be administered, its use in the pharmaceutical preparation is contemplated.

The dose and dosage regimen of agent according to the invention that is suitable for administration to a particular patient may be determined by a physician considering the patient's age, sex, weight, general medical condition, and the specific condition for which the agent is being administered to be treated or prevented and the severity thereof. The physician may also take into account the route of administration, the pharmaceutical carrier, and the agent's biological activity. Selection of a suitable pharmaceutical preparation will also depend upon the mode of administration chosen.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment or prevention therapy. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation or prevention of a particular pathological condition may be determined by dosage concentration curve calculations, as known in the art.

The pharmaceutical preparation comprising the zinc and/or other therapeutic agent may be administered at appropriate intervals, for example, at least twice a day or more until the pathological symptoms are reduced or alleviated, after which the dosage may be reduced to a maintenance level. The appropriate interval in a particular case would normally depend on the condition of the patient. With regard to prevention or reduction of infection, the compositions of the instant invention may be administered in doses at appropriate intervals prior to or immediately after exposure to the microbial pathogen.

Toxicity and efficacy (e.g., therapeutic, preventative) of the particular formulas described herein can be determined by standard pharmaceutical procedures such as, without limitation, in vitro, in cell cultures, ex vivo, or on experimental animals. The data obtained from these studies can be used in formulating a range of dosage for use in human. The dosage may vary depending upon form and route of administration. Dosage amount and interval may be adjusted individually to levels of the active ingredient which are sufficient to deliver a prophylactically effective amount.

### Methods of Treating a Disease or Disorder

The instant invention also encompasses methods of treating, inhibiting, and/or preventing a disease or disorder associated with epithelial barrier by modifying and/or improving epithelial (e.g., endothelial) barriers as described herein. Indeed, as stated hereinabove, the method may comprise administering at least two, at least three, at least four, at least five or more different agents that modify tight junctions and thereby restrict passage across tight junctions (e.g., “tightens” tight junctions and/or structurally modifies the TJs).

The instant invention encompasses methods of inhibiting (e.g., reducing, suppressing), treating, and/or preventing a microbial infection in a subject. Indeed, many microbial pathogens target the tight junction (TJ) seals between epithelial cells of mucosal tissue linings. The TJ is an entry points for local and systemic infection for many microbes such as bacteria and viruses. Typically, microbial pathogens use the tight junctions as docking sites on the mucosal barrier and/or cause a loosening of the TJ barrier, thereby allowing pathogens paracellular access into the stromal region and the vasculature. The compounds described hereinabove induce structural and functional changes in epithelial TJ such that the TJ barrier is improved or, at least, structurally changed. These structural changes render the TJ less susceptible to pathogen docking, TJ loosening, and pathogen infiltration, thereby lessening morbidity.

Microbial pathogens, e.g., viruses, bacteria, fungi, parasites (including dust mites), target the TJ apparatus during the process of infection or even as the means of infection (see, e.g., Guttman et al. (2009) Biochim. Biophys. Acta., 1788:832-41; O'Hara et al. (2008) Front Biosci., 13:7008-21). The microbial pathogens may act to disrupt and make the TJ seals leaky and/or bind to the TJ (e.g., as an entry point into the epithelial cell). Viruses which cause TJ disruption include, without limitation: HIV (Nazli et al. (2010) PLoS Pathog., 6:e1000852), echovirus (Sobo et al. (2011) J. Virol., 85:12376-86), avian influenza virus (Golebiewski et al. (2011) J. Virol., 85:10639-48), rhinovirus (Comstock et al. (2011) J. Virol., 85:6795-808; Yeo et al. (2010) Laryngoscope 120:346-52), human papilloma virus (Kranjec et al. (2011) J. Virol., 85:1757-64), SARS coronavirus (Teoh et al. (2010) Mol. Biol. Cell 21:3838-52), West Nile virus (Verma et al. (2010) Virology 397:130-8; Medigeshi et al. (2009) J. Virol., 83:6125-34), Coxsackie virus (Coyne et al. (2007) Cell Host Microbe 2:181-92; Raschperger et al. (2006) Exp. Cell Res., 312:1566-80) norovirus (Hillenbrand et al. (2010) Scand. J. Gastroenterol., 45:1307-19), herpes virus (e.g., HSV), and hepatitis C virus (HCV). It is clear that certain viruses have evolved to “open up” a mucosal barrier by making the TJ leaky, thereby allowing additional virus to enter the interstitium and systemic circulation. Indeed, many viruses have a PDZ binding domain that seeks to bind to other PDZ-domains, which are found in many TJ-associated proteins (Javier et al. (2011) J. Virol., 85:11544-56). Other microbes also use the tight junction complex as an entry point for invading an epithelial cell. Accordingly, substances that aid in epithelial remodeling or increasing epithelial barrier integrity or function would inhibit viral colonization and/or infection.

Notably, the TJ protein claudin-1 is required for hepatitis C virus (HCV) infection of the epithelial cell (and, thus, the organism) and the TJ protein, occludin, is a co-factor (Ahmad et al. (2011) Virol. J., 8:229; Fofana et al. (2010) Gastroenterology 139:953-64; Liu et al. (2009) J. Virol., 83:2011-4; Ciesek et al. (2011) J. Virol., 85:7613-21. The fact that the extracellular loops of claudin-1 are required for HCV entry indicates that there is interaction outside the cell between HCV and TJ proteins and that this extracellular interaction between virus and TJ is necessary for viral infection (Evans et al. (2007) Nature 446:801-5). Overall, these findings indicate that HCV binds to the TJ as part of its mechanism of entry into the epithelial cell and the organism. Accordingly, modifying the TJ (e.g., to include less claudin-1) would make viral binding and infection less efficient or completely block viral entry, thereby reducing morbidity.

Like viruses, bacterial infections present themselves initially on the mucosal surfaces of barrier tissues (e.g., oral mucosa, nasopharyngeal mucosa, intestinal mucosa, vaginal mucosa, and the like). Certain pathogenic bacteria achieve infection in part by the disruption of TJ barriers. Example of such bacteria include, without limitation: Streptococcus pneumonia (Clarke et al. (2011) Cell Host Microbe., 9:404-14), Haemophilus influenza (Clarke et al. (2011) Cell Host Microbe., 9:404-14), Streptococcus suis (Tenenbaum et al. (2008) Brain Res., 1229:1-17), Bacillus anthracis (Bourdeau et al. (2009) J. Biol. Chem., 284:14645-56), E. coli (Denizot et al. (2012) Inflamm. Bowel Dis., 18:294-304; Strauman et al. (2010) Infect. Immun, 78:4958-64; Roxas et al. (2010) Lab Invest., 90:1152-68), Yersinia enterocolitica (Hering et al. (2011) Lab Invest., 91:310-24), Clostridium difficile (Zemljic et al. (2010) Anaerobe. 16:527-32), Neisseria miningitidis (Schubert-Unkmeir et al. (2010) PLoS Pathog. 6:e1000874, Aeromonas hydrophila (Bucker et al. (2011) J. Infect. Dis., 204:1283-92), Bacteroides fragilis (Obiso et al. (1997) Infect. Immun., 65:1431-9), and Vibrio cholera (Wu et al. (2000) Cell Microbiol., 2:11-7). All of these bacteria involve redistribution of TJ proteins and/or degradation of TJ proteins along with induction of TJ leakiness as part of their mechanism of infection. Notably, Listeria capitalizes on gaps in the epithelial barrier (e.g., at sites of cell extrusion) and then binds to basolaterally-situated E-cadherin as its docking site to the epithelial layer (Pentecost et al. (2006) PLoS Pathog., 2:e3). Accordingly, as with viruses, substances that aid in epithelial remodeling or increasing epithelial barrier integrity would inhibit bacterial colonization and/or infection.

Microbial infections that can be inhibited, treated, and/or prevented by the methods of the instant invention include, without limitation, viral, bacterial, fungal, and parasitic infections. In a particular embodiment, the microbe is selected from the group consisting of HIV, echovirus, influenza virus, rhinovirus, human papilloma virus, SARS, coronavirus, coxsackie virus, norovirus, herpes, and hepatitis C virus. In a particular embodiment, the bacteria is selected from the group consisting of Streptococcus pneumonia, Haemophilus influenza, Streptococcus suis, Bacillus anthracis, E. coli, Yersinia enterocolitica, Clostridium difficile, Neisseria miningitidis, Aeromonas hydrophila, Bacteroides fragilis, Vibrio cholera, and Listeria. In a particular embodiment, the microbial infection is a sexually transmitted disease. The methods of the instant invention comprise administering (directly or indirectly) at least two TJ “tightening” agents of the instant invention to epithelial tissue of the subject. In a particular embodiment, the compound is delivered topically or systemically to the epithelial tissue.

The compositions of the instant invention may be administered before, during, and/or after exposure or risk of exposure to the microbial pathogen. In a particular embodiment, the compositions of the instant invention are administered at least prior to exposure or risk of exposure to the microbial pathogen. The composition may also be administered during exposure to the microbial pathogen. In a particular embodiment, the composition is administered immediately prior to exposure to the microbial pathogen. In certain embodiments, the composition is administered within an hour or an hour, 1-3 hours, or a day prior to exposure to the microbial pathogen.

The methods may also further comprise administering at least one other therapeutic agent or therapy for the inhibition of the microbial infection. In a particular embodiment, the agents for improving epithelial barrier performance are utilized as an adjuvant/compliment to the other therapeutic agent. The other therapeutic agents or therapy may be administered consecutively and/or sequentially with the TJ therapy. In a particular embodiment, one agent may be administered topically while the other agent is administered systemically. In a particular embodiment, the methods further comprise the administration of at least one antimicrobial, antiviral, antifungal, antibacterial, and/or antiparasite compound. Examples of anti-fungal agents include, without limitation: terbinafine hydrochloride, nystatin, amphotericin B, griseofulvin, ketoconazole, miconazole nitrate, flucytosine, fluconazole, itraconazole, clotrimazole, benzoic acid, salicylic acid, and selenium sulfide. Examples of anti-bacterial agents include, without limitation: antibiotics, penicillins, cephalosporins, carbacephems, cephamycins, carbapenems, monobactams, aminoglycosides, glycopeptides, quinolones, tetracyclines, macrolides, fluoroquinolones, and derivatives thereof. Examples of anti-viral agents include, without limitation: amantadine hydrochloride, rimantadin, acyclovir, famciclovir, foscarnet, ganciclovir sodium, idoxuridine, ribavirin, sorivudine, trifluridine, valacyclovir, vidarabin, didanosine, stavudine, zalcitabine, zidovudine, interferon alpha, and edoxudine.

The instant invention encompasses methods of delaying, inhibiting (reducing, suppressing), treating, and/or preventing adenocarcinoma in a subject, particularly a subject with Barrett's esophagus. The instant invention also encompasses methods of delaying, inhibiting, treating, and/or preventing Barrett's esophagus in a subject. For example, the methods may delay or inhibit the progression of Barrett's metaplasia to dysplasia and adenocarcinoma. The methods of the instant invention comprise administering (directly or indirectly) agents of the instant invention (as described hereinabove) to the esophagus of the subject. In a particular embodiment, the agents are delivered orally, topically to the luminal, and/or via an implantable medical device (e.g., a stent). The delivery of the agents has cancer preventative properties and/or accelerates repair of the Barrett's esophagus.

In a particular embodiment, the agents are administered via an implantable device such as a luminal stent, tube, or ring placed within the esophagus (e.g., during an endoscopy). In a particular embodiment, the implantable device is administered to a dyplastic Barrett's esophagus subject. The implantable medical device may be coated with a composition comprising the agents or may elute the composition. In a particular embodiment, the stent is dissolvable or degradable (e.g., a stent that exhibits substantial mass or density reduction or chemical transformation after it is introduced into a subject). In another embodiment, the stent is removable. The stent may be a sustained release device. Examples of esophageal stents include, without limitation, the Boston Scientific Ultraflex™ device, the Medtronic EsophaCoil® device, and the Cook Medical Evolution® device.

The methods may also further comprise the administration of at least one other therapeutic agent or therapy for the treatment of Barrett's esophagus. This other agent may act topically or systemically. The other therapeutic agents of therapy may be administered consecutively and/or sequentially with the TJ tightening therapy. Examples of Barrett's esophagus treatment methods include, without limitation, surgical treatment (e.g., of high-grade dysplasia), endoscopic ablation therapy (e.g., for removal of high-grade dysplasia in the esophagus), chemical ablation (e.g., via photodynamic therapy (PDT) for dysplasia (e.g., PDT with porfimer sodium (Photofrin®))), thermal ablation, mechanical ablation, endoscopic mucosal resection, pharmaceutical treatment, or a combination of any of these therapies. Examples of pharmaceutical therapies include the following. WO 2005/012275 describes methods for treating or preventing Barrett's esophagus comprising administering to a mammal in need of such treatment of prevention an effective dose of at least one CCK2 (cholecystokinin) modulator. Further, WO 2005/079778 describes the use of a retinoic acid antagonist in the manufacture of a medicament for the treatment or prevention of Barrett's esophagus. Buttar et al. (Gastroenterology (2002) 122:1101-1112) describe the chemoprevention of esophageal adenocarcinoma by COX-2 inhibitors in an animal model of Barrett's esophagus. U.S. Patent Application Publication No. 2009/0306049 describes methods of treating and inhibiting Barrett's esophagus using Notch pathway inhibitors. In a particular embodiment, the methods further comprise the administration of at least one chemotherapeutic agent.

The methods may also comprise the administration of at least one analgesic and/or anesthetic, particularly when administering the agents of the instant invention via an implantable medical device such as a stent. In a particular embodiment, the stent further comprises at least one analgesic and/or anesthetic when the TJ tightening agents containing stent is administered after surgery or treatment to remove the damaged section of the esophagus (e.g., ablation). As stated hereinabove, the ablation procedures to remove Barrett's esophagus tissue are painful. The presence of at least one analgesic and/or anesthetic with the stent reduces the pain experienced by the subject and makes it more likely the subject will consent to undergoing the procedure again, if needed.

The methods of the instant invention may also comprise detecting and monitoring the presence of Barrett's esophagus in the subject. Barrett's esophagus may be detected and monitored by endoscopy and/or biopsy as described hereinabove. Barrett's esophagus may be detected and monitored before, during, and/or after treatment.

The instant invention also encompasses methods of delaying, inhibiting (reducing, suppressing), treating, and/or preventing other endothelial diseases such as, without limitation, colitis, Crohn's disease, HIV enteropathy, accumulation of lung water, multi-organ failure, colic, gastroesophageal reflux disease (GERD), non-erosive reflux disease (NERD), Celiac disease, and/or inflammatory bowel disease in a subject. The methods comprise administering the TJ tightening agents to a subject (as described hereinabove). In a particular embodiment, the method comprises administering zinc and another barrier enhancing agent. In a particular embodiment, the subject is an infant and the disorder is colic.

### Screening Methods

The instant invention also encompasses methods of screening for synergistic combinations of TJ tightening/improving agents. In a particular embodiment, the method comprises contacting an epithelial layer with at least two different agents which improve epithelial barrier function (as described above) and measuring the leakage of the epithelial layer (e.g., measuring electrical resistance), wherein a decrease in leakage compared to the leakage observed with the agents individually, indicates a synergy with the tested agents. In a particular embodiment, the decrease in leakage observed is greater than the combined (additive) decrease of the agents individually. The epithelial layer may be cell layer associated with an epithelial disease. In a particular embodiment, the agents are selected from the group consisting of zinc, amino acids (e.g., glutamine and arginine), rapamycin, berberine, flavonoids (e.g., quercetin), short chain and omega-3 fatty acids and lipids (e.g., linolenic acid, lipoic acid, and butyrate), cruciform-derived compounds (e.g., sulfuraphane), anthocyanins (e.g., cyanidin), stilbenoids (e.g., resveratrol), indole, selenium, and niacin. In a particular embodiment, at least one of the agents is zinc (or a zinc containing substrate as described herein). In a particular embodiment, neither agent is zinc. In a particular embodiment, the method comprises measuring the leakage of at least one of electrolyte (e.g., salts), small non-electrolye (e.g., mannitol or lactulose), large non-electrolye (e.g., inulin), and/or charged compound/molecule.

The above screening methods can be used to identify agents which would work synergistically against a particular disease or disorder (as described above). For example, if a disease or disorder is associated with the colon, then colonic epithelial layers may be used in the above methods to identify synergistic combinations of agents for improving the function of the colonic epithelial barrier. In a particular embodiment, the above screening method can be performed in the presence of a microbe (e.g., pathogen; as described above) in order to identify synergistic combinations of agents for inhibiting (e.g., reducing, suppressing), treating, and/or preventing a microbial infection in a subject. For example, the above screening methods may be performed in the presence of a bacterial toxin to observe the leakage of the epithelial layer to the bacterial toxin, particularly where the epithelial layer is the site of bacterial infection. As another example, in the context of HIV or other STD-associated viruses, the above screening methods can be performed with vaginal epithelial cells and/or in the presence of the virus to observe leakage (e.g., to the virus).

### DEFINITIONS

The following definitions are provided to facilitate an understanding of the present invention:

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y., 1980; and Kibbe, et al., Eds., Handbook of Pharmaceutical Excipients, American Pharmaceutical Association, Washington.

As used herein, the term “subject” refers to an animal, particularly a mammal, particularly a human.

Terms that refer to being “anti” a type of target organism (e.g., antimicrobial, antiviral, antifungal, antibacterial, antiparasite) refers to having any deleterious effects upon those organisms or their ability to cause symptoms in a host or patient. Examples include, but are not limited to, inhibiting or preventing infection, inhibiting or preventing growth or reproduction, killing of the organism or cells, and/or inhibiting any metabolic activity of the target organism. The term “antimicrobial” refers to any substance or compound that when contacted with a living cell, organism, virus, or other entity capable of replication, results in a reduction of growth, viability, or pathogenicity of that entity. As used herein the term “antibiotic” refers to a molecule that inhibits bacterial growth or pathogenesis.

As used herein, the term “prevent” refers to the prophylactic treatment of a subject who is at risk of developing a condition (e.g., microbial pathogen infection) resulting in a decrease in the probability that the subject will develop the condition.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain without a loss of consciousness). Analgesics include opioid analgesics (e.g., codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine) and non-opiate analgesics (e.g., NSAIDs such as salicylates (e.g., aspirin, methyl salicylate, and diflunisal); arylalkanoic acids (e.g., indomethacin, sulindac, diclofenac, and tolmetin); N-arylanthranilic acids (e.g., fenamic acids, mefenamic acid, and mecflofenamate); oxicams (e.g., piroxicam and meloxicam); coxibs (e.g., celecoxib, rofecoxib, valdecoxib, parecoxib, and etoricoxib); sulphonanilides (e.g., nimesulide); naphthylalkanones (e.g., nabumetone); anthranilic acids (e.g., pyrazolidinediones and phenylbutazone); proprionic acids (e.g., fenoprofen, flurbiprofen, ibuprofen, ketoprofen, naproxen, and oxaprozin); pyranocarboxylic acids (e.g., etodolac); pyrrolizine carboxylic acids (e.g., ketorolac); and carboxylic acids.

As used, the term “anesthetic” refers to an agent that produces a reversible loss of sensation in an area of a subject's body. An agent may act as both an analgesic and an anesthetic. Anesthetics include, without limitation, benzocaine, benzyl alcohol, bupivacaine, butamben picrate, chlorprocaine, cocaine, dibucaine, dimethisoquin, dyclonine, etidocaine, hexylcaine, ketamine, lidocaine, mepivacaine, phenol, pramoxine, procaine, tetracaine, salicylates, ropivacaine, prilocaine, and xylocaine.

As used herein, the term “metaplasia” refers to the replacement of one differentiated cell type with another differentiated cell type. Metaplasia is not directly considered carcinogenic.

The term “implantable medical device” refers to any medical device placed inside the human body. The placement of such a device may occur in a body lumen of the patient, such as the esophagus.

As used herein, the term “small molecule” refers to a substance or compound that has a relatively low molecular weight (e.g., less than 10,000, particularly less than 4,000 or less than 2,000).

The following examples are provided to illustrate various embodiments of the present invention. They are not intended to limit the invention in any way.

### Example 1

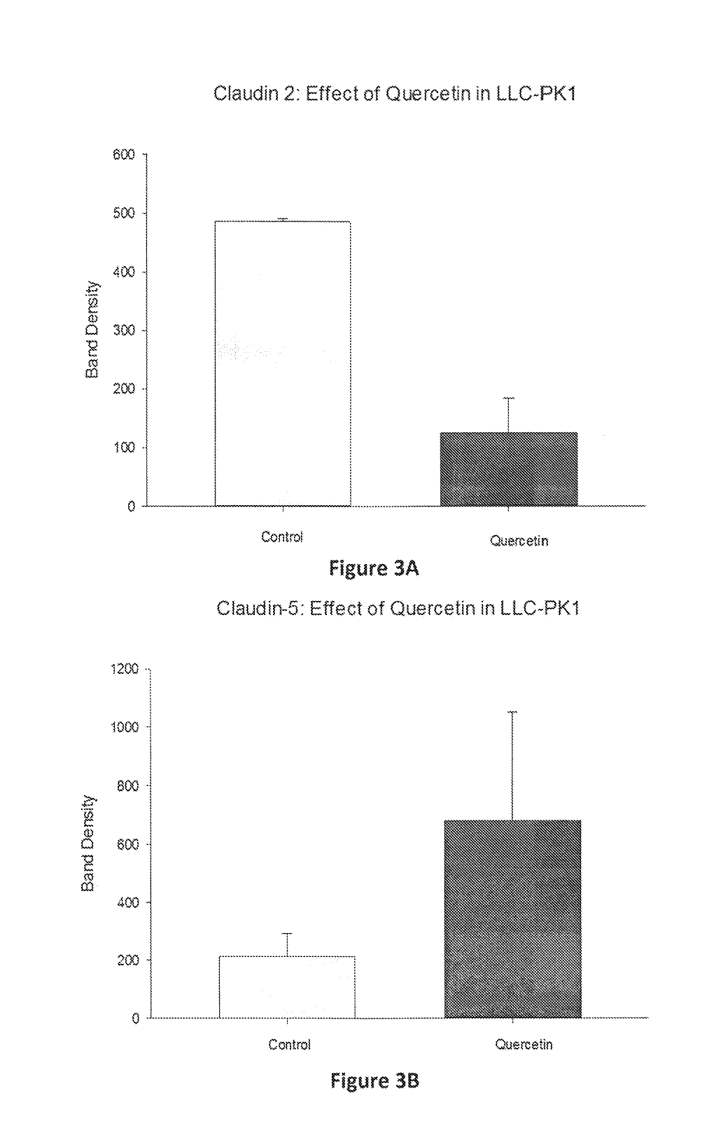

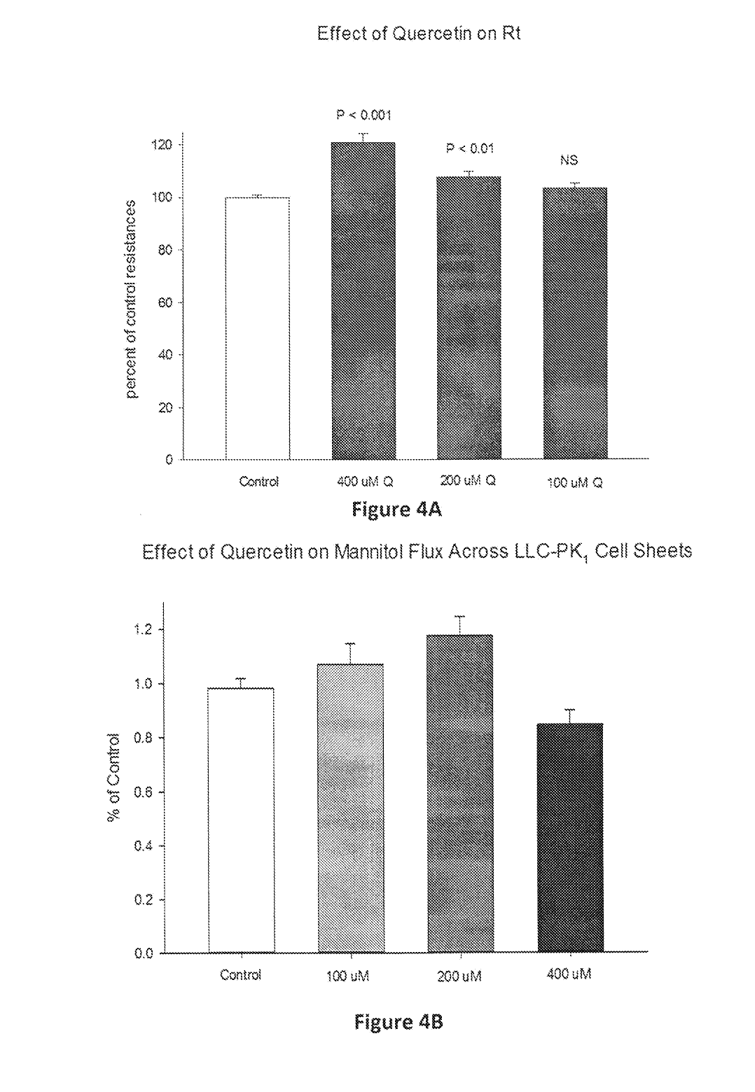

LLC-PK1 is a porcine cell line model of the kidney proximal tubule. As seen in FIGS. 3A and 3B, the incubation of LLC-PK1 cells with 400 μM quercetin resulted in dramatic changes in the expression of Claudin 2 (downregulated) and Claudin 5 (upregulated), respectively. The incubation of LLC-PK1 cell layers with quercetin also resulted in increased transepithelial resistance and a decrease in mannitol leaking across the cell layer (see FIGS. 4A and 4B).

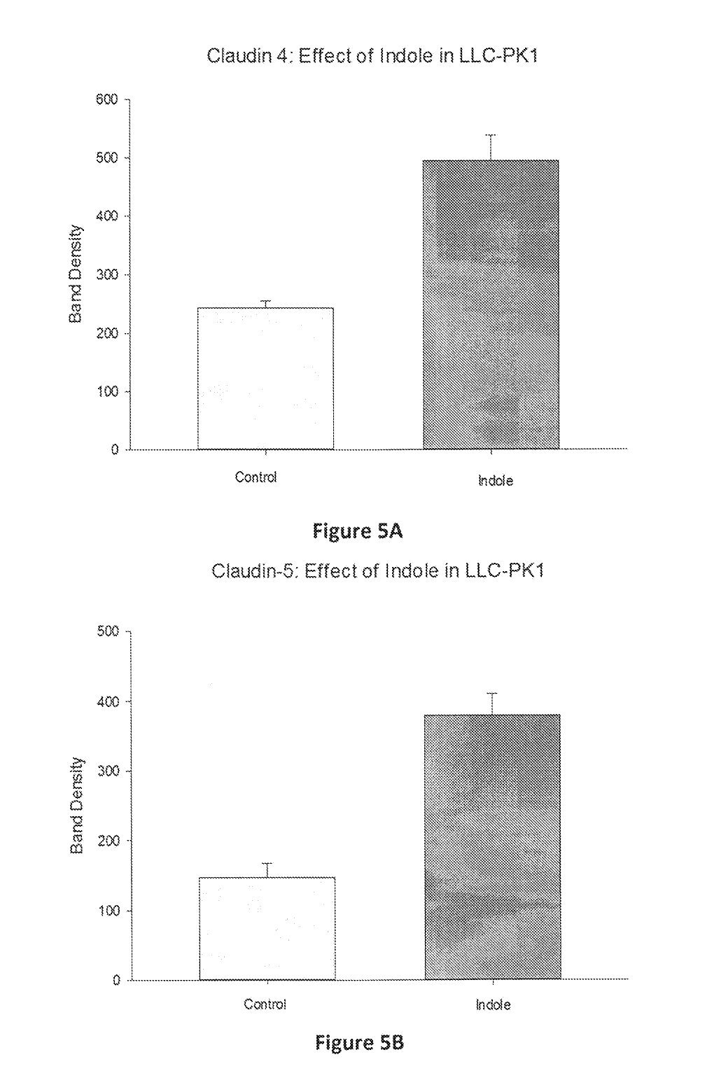

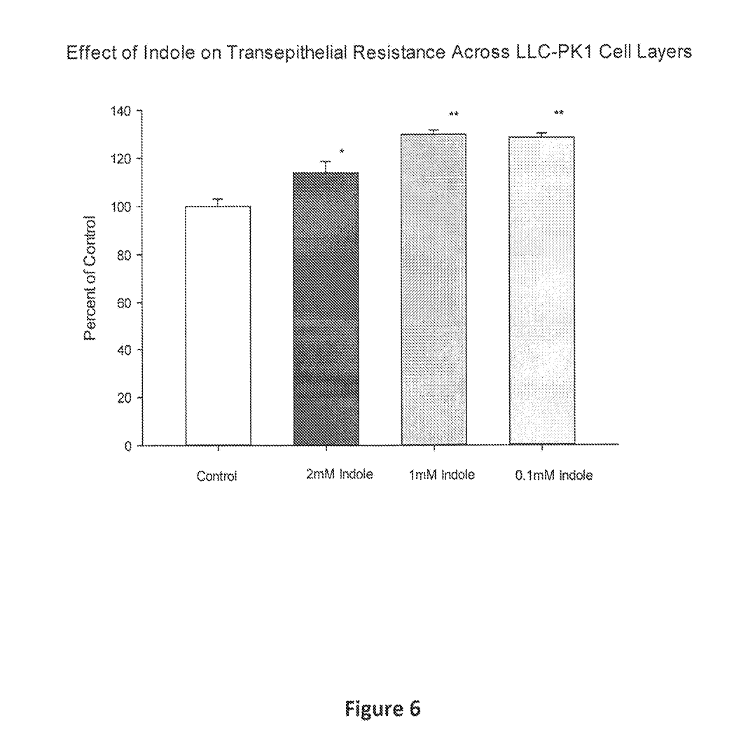

As seen in FIGS. 5A and 5B, the incubation of LLC-PK1 cells with indole resulted in dramatic changes in the expression of Claudin 4 (upregulated) and Claudin 5 (upregulated), respectively. Notably, indole had a less than 50% effect on claudin 1, 2, 3, or 7, tricellulin, or occludin, which are all tight junction barrier proteins. The incubation of LLC-PK1 cell layers with indole also resulted in increased transepithelial resistance across the cell layer (see FIG. 6).

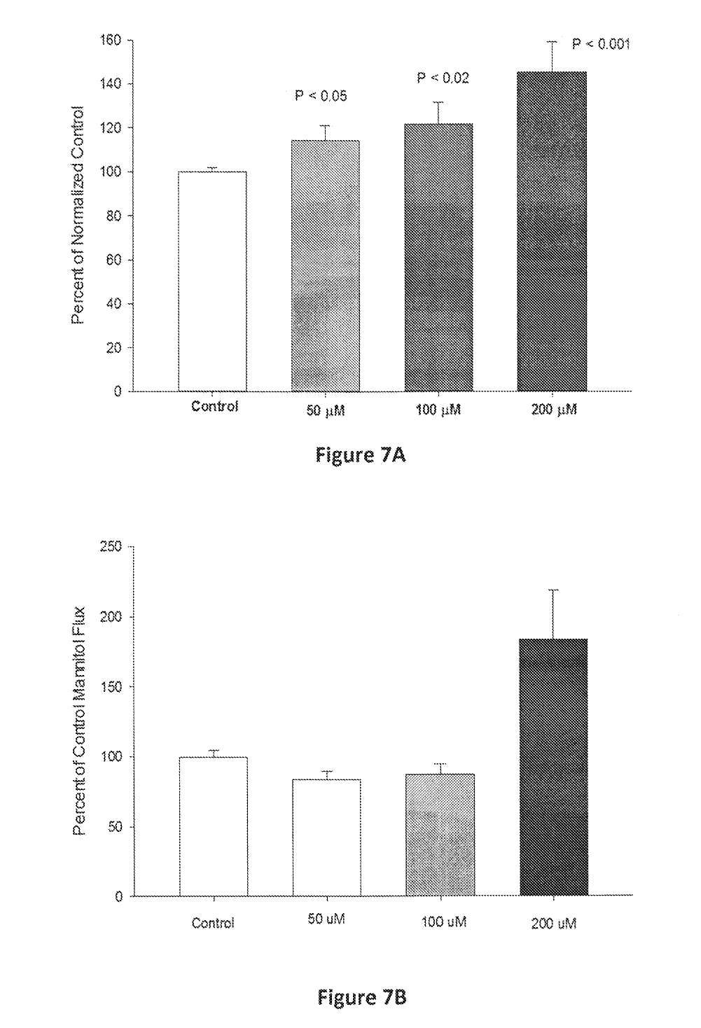

The incubation of LLC-PK1 cell layers with zinc also resulted in increased transepithelial resistance and a decrease in mannitol leaking across the cell layer (see FIGS. 7A and 7B).

Table 1 provides a summary of the effects of zinc, indole, and quercetin on LLC-PK1 cell layers.

TABLE 1
Barrier Enhancing Agent Effects on LLC-PK1 Cell Layers.

| | | | 400 μM |
|---|---|---|---|
| | 100 μM Zn | 1 mM Indole | Quercetin |
| Transepithelial | Incr > 40% | Incr 30% | Incr > 20% |
| Resistance | | | |
| Mannitol Flux | Decr > 20% | NC | Decr > 20% |
| Short Circuit | NC | NC | NC |
| Current | | | |
| Occludin | NC | NC | NC |
| Tricellulin | NC | Decr 10% | Decr 15% |
| Claudin-1 | NC | Decr 30% | NC |
| Claudin-2 | NC | Decr 40% | Decr 75% |
| Claudin-3 | NC | Decr 20% | Decr 30% |
| Claudin-4 | NC | Incr 100% | Incr 75% |
| Claudin-5 | NC | Incr 150% | Incr 100% |
| Claudin-7 | NC | NC | Incr 200% |

NC = no change.

### Example 2

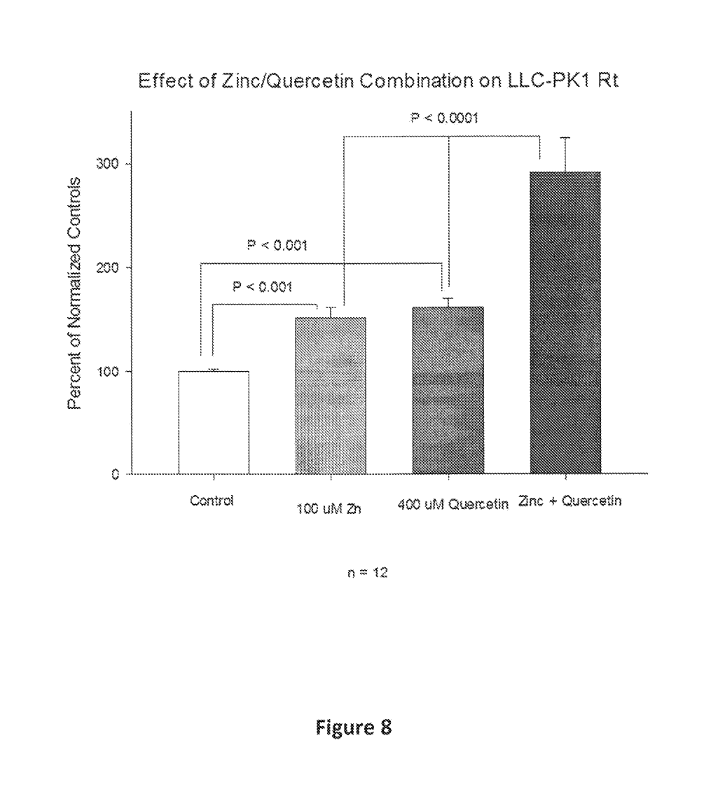

The effect of zinc and/or quercetin on the electrical resistance across LLC-PK1 renal epithelial cell layers was tested. Briefly, LLC-PK1 renal epithelial cell layers were treated with either 100 μM zinc alone, 400 μM quercetin alone; or both agents together. The stimulation of transepithelial electrical resistance (Rt) by maximally effective concentrations of zinc alone and quercetin alone was in the range of 50-60% of control levels. However when cell layers were treated with the two compounds together at the aforementioned concentrations, a 200% stimulation of transepithelial electrical resistance was observed, demonstrating more than a simple additive effect (i.e., synergy) compared to the compounds individually. The results shown in FIG. 8 represent the mean+/− standard error for 12 cell layers. P values are for one-sided Student's t tests.

The effects of zinc or quercetin alone or in combination on tight junctional proteins were also studied. The more than additive effect of zinc with quercetin on transepithelial electrical resistance of LLC-PK1 cell layers may, in part, owe its effect to actions on certain tight junctional proteins. Notably, the combination of zinc and quercetin produced a greater increase of tight junctional claudin-7 levels than for either nutritional agent alone. While the increase in claudin-7 levels was mostly additive, the nutritional agents may also shift certain claudins out of tight junctional complexes (e.g., into the cell's cytoplasm), thereby effecting tight junctions but not the overall cellular content of the claudin.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of altering the structure of tight junctions and/or improving epithelial barrier function comprising administering at least two agents which decrease leakage across tight junctions to the epithelial tissue of a subject.

2. The method of claim 1, wherein said agents are selected from the group consisting of zinc, amino acids, rapamycin, berberine, flavonoids, isoflavonoids, lycopenes, short chain fatty acids, omega-3 fatty acids, indole, selenium, stilbenoids, anthocyanins, cruciform organo-sulfur compounds, and niacin.

3. The method of claim 1, wherein at least one agent is zinc.

4. The method of claim 3, wherein said zinc is a zinc salt.

5. The method of claim 4, wherein said zinc salt is zinc gluconate.

6. The method of claim 3, wherein at least one other agent is quercetin.

7. The method of claim 1, wherein said agents are administered topically.

8. The method of claim 7, wherein said agents are administered to the skin.

9. The method of claim 7, wherein said agents are administered to oral, colorectal, bladder, uterine, nasal, vaginal, penile, nasopharyngeal, buccal, or intestinal epithelial or mucosa.

10. The method of claim 1, wherein said agents act topically or systemically.

11. A method of inhibiting a disease or disorder associated with epithelial barrier leakage or traversal of the epithelial barrier, said method comprising administering at least two agents which decrease leakage across tight junctions to the epithelial tissue of a subject.

12. The method of claim 11, wherein said disease or disorder is a microbial infection.

13. The method of claim 11, wherein said disease or disorder is an epithelial disease.

14. A method of screening for synergistic combinations of agents for decreasing leakage across tight junctions, said method comprising:
a) contacting an epithelial layer with at least two different agents for decreasing leakage across tight junctions; and
b) measuring the leakage of the epithelial layer, wherein a decrease in the leakage measured in step b) compared to the leakage observed with the agents individually indicates a synergy with the tested agents.

15. The method of claim 14, wherein the agents are selected from the group consisting of zinc, amino acids, rapamycin, berberine, flavonoids, isoflavonoids, lycopenes, stilbenoids, anthocyanins, cruciform organo-sulfur compounds, short chain fatty acids, omega-3 fatty acids, indole, selenium, and niacin.

16. The method of claim 14, wherein step b) comprises measuring the leakage of at least one of electrolyte, small non-electrolye, large non-electrolye, or charged compound/molecule.

17. The method of claim 14, wherein said epithelial layer is associated with a disease or disorder.
